(19) United States
(12) Patent Application Publication
(10) Pub. No.: US 2005/0070852 A1
(43) Pub. Date: Mar. 31, 2005

(54) Percutaneous endoscopic gastrotomy tube holder

(76) Inventor: Vivian A. Wright, Coral Gables, FL (US)

Correspondence Address: Pepper Hamilton LLP;Firm 21269, 50th Floor, 500 Grant Street, One Mellon Center, Pittsburgh, PA, 15219, US

(21) Appl. No.: 10/670,435

(22) Filed: Sep. 25, 2003

(51) Int. Cl. A61M 5/32

(52) U.S. Cl. 604/180

(57) ABSTRACT

A medical device and methods of using the medical device to hold a percutaneous endoscopic gastrotomy (PEG) tube. The medical device includes a front side and a back side. The front side includes a left section, a middle section, and a right section. The left section and the right section each include a refastenable material. The back side includes an adhesive section and a non-adhesive section. The adhesive section adheres the device to the skin of a recipient. A PEG tube placed in the middle section is secured within the device by applying pressure to the non-adhesive section until the refastenable material of the left and right sections engage. Conversely, the tube may be removed without affecting the adhesion of the device to the recipient or the integrity of the tube by disengaging the left and right sections of the device.

### FIELD OF INVENTION

The present invention relates generally to an apparatus in the field of gastrointestinal therapy and more particularly to an apparatus for protecting a Percutaneous Endoscopic Gastrotomy (PEG) tube and stoma when fitted to a recipient.

### BACKGROUND OF THE INVENTION

Previous apparatuses have been developed for securing medical tubes in direct contact with a recipient's skin. These apparatuses may use an adhesive, which may be hypoallergenic, to affix the holding apparatus to the recipient's skin. In addition, the apparatuses must be secured around the tube itself. Some of these apparatuses hold a tube by applying an adhesive portion of the apparatus directly to the tube. Other apparatuses hold a tube by adhering opposing sides of the apparatus.

Applying an adhesive portion of a holding apparatus to a tube has numerous disadvantages. For instance, by directly adhering the tube to the holder, the range of movement of the tube may be limited. Furthermore, if medical personnel desire to remove the tube from the holder, the tube must first be removed from the adhesive portion of the holder. This may cause stress and damage to the tube. In addition, the adhesive portion of the holder may lose some of its adhesiveness each time the tube is removed from the holder causing the holder to lose the ability to securely hold a tube over time.

What is needed is a medical tube holding apparatus that allows the tube to be secured to a recipient and to the tube in such a manner that removal of the tube from the holder does not cause damage to the tube. Furthermore, a PEG tube holding apparatus is needed that allows the tube to have a range of motion within the holder so that a recipient may not be restricted in his or her movement. Moreover, the PEG tube holding apparatus must be able to be produced at a low cost.

### SUMMARY OF THE INVENTION

The present invention is directed to solving one or more of the aforementioned problems.

In a preferred embodiment of the present invention, an apparatus for holding a percutaneous endoscopic gastrotomy (PEG) tube includes a front side and aback side. The front side includes a left section, a middle section, and a right section. The left and right sections each have a surface at least partially covered by a refastenable material, such as VELCRO®. The middle section has a surface including a first material. In an embodiment, the first material is cotton. The first material may cover substantially all of the middle section.

The back side includes an adhesive section and a non-adhesive section. The adhesive section has a surface at least partially covered by a hypoallergenic adhesive. In a preferred embodiment, a protective cover covers the hypoallergenic adhesive. The hypoallergenic adhesive may cover substantially all of the surface of the adhesive section. Alternately, the hypoallergenic adhesive is aligned in one or more strips on the surface of the adhesive section. The non-adhesive section has a surface including a second material. In an embodiment, the second material is cotton. The second material may cover substantially all of the non-adhesive section.

In a preferred embodiment, the apparatus has a notch removed from a first portion of the non-adhesive section and the one or more sections of the front side opposed to the first portion of the non-adhesive section.

In a preferred embodiment, a method of applying a PEG holder to a recipient in order to hold a PEG tube includes first adhering the PEG holder to the recipient. The PEG holder includes a front side and a back side. The front side includes a left section, a middle section, and a right section. The left and right sections each have a surface at least partially covered by a refastenable material. The back side has a surface partially covered by a hypoallergenic adhesive for adhering the PEG holder to the recipient. The method also includes placing the PEG tube in the middle section and affixing the refastenable material of the left section to the refastenable material of the right section. In an embodiment, the method further includes removing a protective cover covering the hypoallergenic adhesive before adhering the PEG holder to the recipient. In an embodiment, placing the PEG tube includes aligning the PEG tube such that a receiving end of the PEG tube extends substantially vertically upward.

In a preferred embodiment, a method of removing a PEG tube from a PEG holder attached to a recipient includes first unfastening a PEG holder. The PEG holder includes a front side and a back side. The front side includes a left section, a middle section, and a right section. The left and right section each have a refastenable material. The back side includes a surface that is partially covered by a hypoallergenic adhesive. The back side is initially adhered to a recipient. Unfastening the PEG holder includes unfastening the left section of the PEG holder from the right section of the PEG holder. The method also includes removing the PEG tube from the middle section. In an embodiment, the method further includes removing the back side of the PEG holder from the recipient.

This apparatus provides several advantages, including an increase in patient comfort and an increase in the reliability and lifespan of a PEG tube as it is less likely to be compromised. In addition, a higher level of medical care may result an enable stoma health. Another advantage of the tube holder is that it may be manufactured from readily available materials, utilizing common manufacturing technologies and techniques.

### BRIEF DESCRIPTION OF THE DRAWINGS

In part, other aspects, features, benefits and advantages of the embodiments of the present invention will be apparent with regard to the following description, appended claims and accompanying drawings where:

FIG. 1 illustrates a front view of a preferred embodiment of the present invention.

FIG. 2 illustrates a back view of a preferred embodiment of the present invention.

FIG. 3 illustrates a front view of an exemplary PEG holder as it envelopes a PEG according to an embodiment of the present invention.

FIG. 4 illustrates a front view of an exemplary embodiment of the present invention as attached to the body of a user and folded into an enveloped PEG holder.

### DETAILED DESCRIPTION OF THE INVENTION

Before the present compositions and methods are described, it is to be understood that this invention is not limited to particular compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a,” “an” and “the” include plural references unless the context clearly dictates otherwise. Thus, for example, reference to a “PEG holder” is a reference to one or more PEG holders and equivalents thereof known to those skilled in the art, and so forth. Unless defined otherwise, all technical and scientific terms used herein have the same meanings as commonly understood by one of ordinary skill in the art. Although any methods, devices and material similar or equivalent to those described herein can be used in the practice of testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

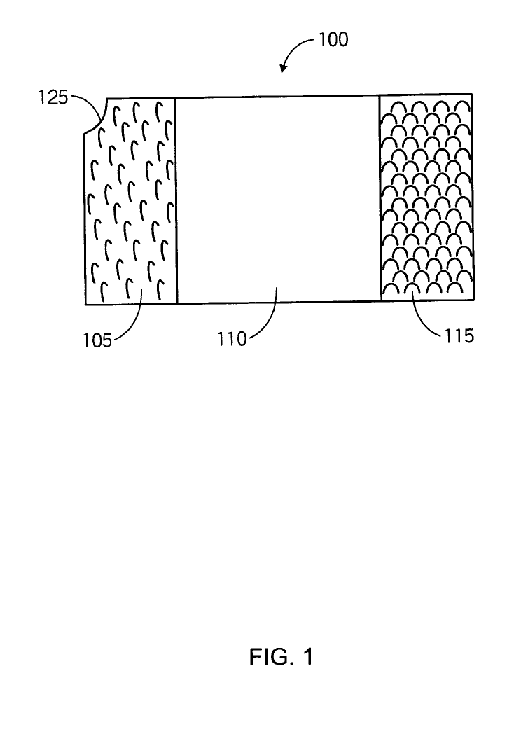

The present invention provides a PEG holder that functions as a safe anchor for a PEG. FIG. 1 illustrates a front side of a preferred embodiment of the present invention. The PEG holder 100 may be a pad with a substantially rectangular perimeter having seamed and/or sealed perimeter edges. In a preferred embodiment, a corner of the PEG holder 100 may have a notch 125 cut out of it. The notch 125 may also be seamed and/or sealed. In a preferred embodiment, the PEG holder 100 may be approximately four inches long by approximately two inches wide by approximately one-eighth of an inch thick. The surface of the front side of the PEG holder 100 may be divided into three sections lengthwise, including a left section 105, a middle section 110 and a right section 115. The left section 105 and the right section 115 may each encompass approximately one-fourth of the length of the PEG holder 100. Each of the left section 105 and the right section 115 may each be covered with a material that allows for repetitive attachment and disengagement of the two sections, such as VELCRO®. The middle section 110 may encompass approximately one-half of the length of the PEG holder 100, and may be covered in an absorbent material, such as cotton.

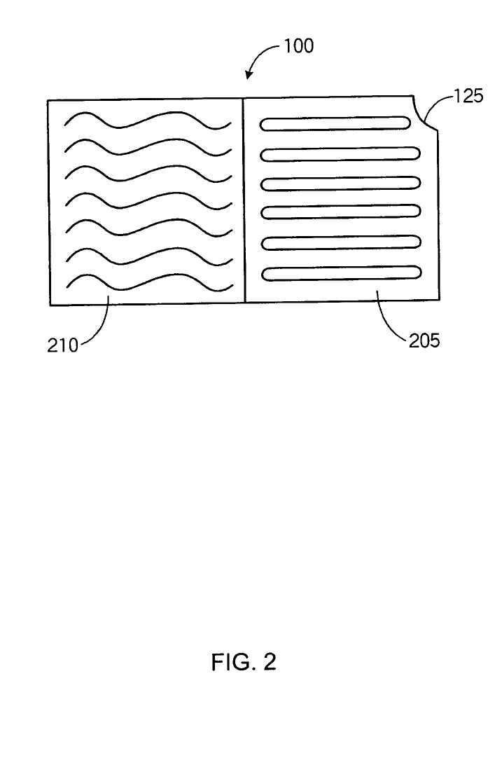

FIG. 2 illustrates a back side of a preferred embodiment of the present invention. The back side of a PEG holder 100 may also be referred to as a contact side. The back side of the PEG holder 100 may be sectioned in two substantially equal sized lengthwise sections. A non-adhesive section 205 may be covered in an absorbent material, preferably cotton. An adhesive section 210 may be at least partially covered with a hypoallergenic adhesive. In a preferred embodiment, the adhesive may be applied to substantially all of the adhesive section 210. In an alternate embodiment, the adhesive may be applied in adhesive strips in any orientation within the adhesive section 210. Prior to adhesion to a recipient, the adhesive section 210 may have a protective cover (not shown). Once the protective cover is removed, the adhesive properties may be revealed and introduced to the recipient. A corner of the non-adhesive section 205 may have a tab extending up from the edge.

The PEG holder 100 may ensure that a user's PEG and stoma, a surgically created opening in the stomach in which the PEG is placed, are protected. The PEG holder 100 may be made of a thin absorbent material, preferably cotton. The PEG holder 100 may be reusable and inexpensive to manufacture. It may also be pre-assembled, lightweight, portable and comfortable for the user, ensure the health of the stoma, and protect the condition of the PEG.

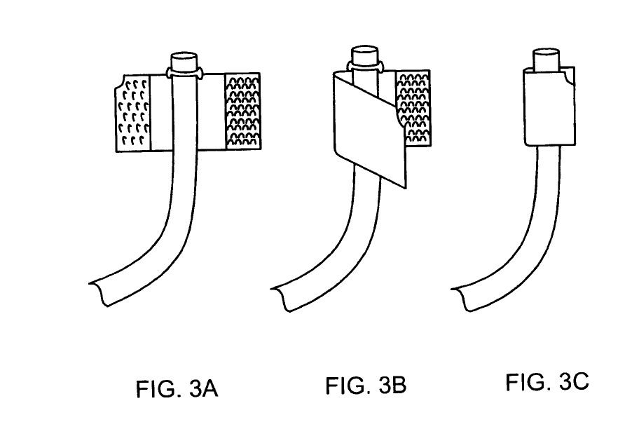

FIGS. 3A-3C illustrate front views of an exemplary PEG holder 100 as it is attached for use according to an embodiment of the present invention. A preferred method of using the PEG holder 100 will now be described. The protective cover from the adhesive section 210 of the back side of the PEG holder 100 may be removed. The adhesive section 210 may be placed against the skin of a recipient, preferably in a location near a stoma. By pressing gently on the front side of the PEG holder 100, the adhesive section 210 may adhere to the skin of the recipient, while the non-adhesive section 205 of the back side of the PEG holder 100 may remain free from the skin. A PEG may be placed in the middle section 110 of the front side of the PEG holder 100 with the PEG's valve facing substantially in a vertically upward direction, as shown in FIG. 3A. The tube of the PEG may be held against the middle section 110 of the PEG holder 100 by a user while the non-adhesive section 205 is folded around the PEG, as shown in FIG. 3B. The non-adhesive section 205 may be pressed until the VELCRO® on the left section 105 of the front side is coupled with the VELCRO® on the right section 115, as shown in FIG. 3C. At this point, the PEG may be secure within the enveloping PEG holder 100.

When a user, caregiver, or health care professional needs to administer liquid foods or mendicants to the recipient, the PEG may be accessed by unfastening the PEG holder 100. The PEG holder 100 may be unfastened by inserting a finger between the left section 105 and the right section 115 of the PEG holder 100. The user may then gently pull the non-adhesive section 205 away from the adhesive section 210 so that the VELCRO® on the left section 105 and the right section 115 disengage. In a preferred embodiment, a notch 125 is provided on the non-adhesive section 205 and the opposing section or sections of the front side to aid the disengagement of the left section 105 from the right section 115. Once the left section 105 and the right section 115 are disengaged, the PEG may be removed from the middle section 110 of the PEG holder 100.

When removing the PEG holder 100 from the skin of a recipient, the PEG must first be removed as described in the preceding paragraph. The user, caregiver, or health care professional may then gently pull the adhesive section 210 away from the skin until it is disengaged.

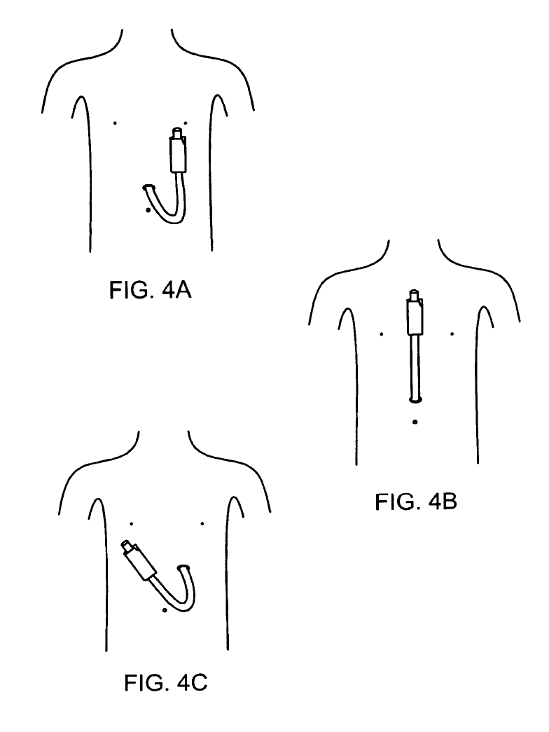

FIGS. 4A-4C illustrate front views of an exemplary embodiment of the present invention as it may be attached to the body of a user and folded into the enveloped PEG holder form. FIGS. 4A-4C represent plausible positionings of the PEG holder 100 on the body of a recipient to ensure that gravitational pressure from the PEG on the stoma is avoided. In a preferred embodiment, the PEG valve and fluid delivery end of the PEG may be pointed in a substantially vertically upward direction to prevent leakage of remnant fluid from the PEG. FIG. 4B may represent a most preferred embodiment since remnant fluid may not gather within the PEG.

1. An apparatus for holding a percutaneous endoscopic gastrotomy (PEG) tube proximate to a recipient comprising:
a front side comprising:
a left section having a surface at least partially covered by a refastenable material,
a right section having a surface at least partially covered by the refastenable material, and
a middle section having a surface including a first material; and
a back side comprising:
an adhesive section having a surface at least partially covered by a hypoallergenic adhesive, and
a non-adhesive section having a surface including a second material.

2. The apparatus of claim 1 wherein the refastenable material is VELCRO®.

3. The apparatus of claim 1 wherein the first material is cotton.

4. The apparatus of claim 1 wherein the first material covers substantially all of the middle section.

5. The apparatus of claim 1 wherein the second material is cotton.

6. The apparatus of claim 1 wherein the second material covers substantially all of the non-adhesive section.

7. The apparatus of claim 1 wherein the hypoallergenic adhesive covers substantially all of the adhesive section.

8. The apparatus of claim 1 wherein the hypoallergenic adhesive includes one or more strips on the surface of the adhesive section.

9. The apparatus of claim 1 wherein the hypoallergenic adhesive is covered by a protective cover.

10. The apparatus of claim 1 having a notch removed from a first portion of the non-adhesive section and the one or more sections of the front side opposed to the first portion of the non-adhesive section.

11. A method of applying an PEG holder to a recipient in order to hold a PEG tube comprising:
adhering a back side of the PEG holder to the recipient, wherein the PEG holder includes a front side and a back side, wherein the front side includes a left section having a surface at least partially covered by a refastenable material, a right section having a surface at least partially covered by the refastenable material, and a middle section, and wherein the back side includes a surface at least partially covered by a hypoallergenic adhesive;
placing the PEG tube in the middle section; and
affixing the refastenable material of the left section to the refastenable material of the right section.

12. The method of claim 11 wherein placing the PEG tube further comprises aligning the PEG tube such that a receiving end of the PEG tube extends substantially vertically upward.

13. The method of claim 11 further comprising, before the adhering step, removing a protective cover covering the hypoallergenic adhesive.

14. A method of removing a PEG tube from a PEG holder attached to a recipient comprising:
unfastening a refastenable material of a left section of a PEG holder from a refastenable material of a right section of a PEG holder, wherein the PEG holder includes a front side and a back side, wherein the front side includes a left section having the refastenable material, a right side having the refastenable material, and a middle section, and wherein the back side is adhered to a recipient; and
removing the PEG tube from the middle section.

15. The method of claim 14 further comprising removing the back side of the PEG holder from the recipient.
